(19) United States
(12) Patent Application Publication
(10) Pub. No.: US 2026/0069218 A1
(43) Pub. Date: Mar. 12, 2026

(54) MANAGEMENT DEVICE FOR DISPLAYING GLUCOSE INFORMATION

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 19/106,212

(22) PCT Filed: Sep. 15, 2022
(86) PCT No.: PCT/CN2022/119084

§ 371 Date: Feb. 25, 2025

(51) Int. Cl. A61B 5/00; A61B 5/145

(52) U.S. Cl. CPC A61B 5/7435; A61B 5/14532; A61B 5/7282; A61B 5/743; A61B 5/7445; A61B 5/746

(57) ABSTRACT

Disclosed is a management device for displaying glucose information. The screen of the management device is divided into the plurality of display areas, at least comprising a first display area for displaying the status of the management device, a second display area for displaying real-time glucose information, and a third display area for displaying historical glucose concentration information. The user can intuitively and easily get the glucose information in the body.

### TECHNICAL FIELD

The invention mainly relates to the field of medical device, in particular to a management device and method for displaying glucose information.

### BACKGROUND

The pancreas in the healthy human body can automatically monitor the glucose level and automatically secrete required amount of insulin/glucagon. For the type 1 diabetes patient, the pancreas does not function properly and cannot secrete enough insulin for the body. Therefore, type 1 diabetes is the metabolic disease caused by abnormal pancreatic function, and diabetes isa chronic disease. At present, there is no cure for diabetes with medical technology. The onset and development of diabetes and its complications can only be controlled by stabilizing glucose.

Diabetics need to have their glucose measured before they inject insulin into the body. At present, most of the testing methods can continuously measure glucose level and transmit the data to the remote device in real-time for the user to view. This method is called Continuous Glucose Monitoring (CGM).

The remote equipment needs to visually process the received glucose data, that is, analyze, display and prompt the data in the manner acceptable to the user. Therefore, it is necessary to develop the personal diabetes manager (PDM) device used in conjunction with the CGM device.

Generally, when the PDM leaves the factory, the manufacturer has set various analysis, display and prompt functions of the PDM. With the continuous development of diabetes monitoring technology, the user's demand for diabetes monitoring becomes more and more refined and perfect. Therefore, the PDM needs to open some data processing permissions for the user to operate or set independently, such as high/low glucose alarm threshold, diet, exercise, sleep, pregnancy, pathological treatment and other personalized settings. With the improvement of the function of PDM, the contents and interfaces displayed on the screen gradually increase. How to reasonably arrange the contents and interfaces displayed on the screen also needs to be considered in the design process of PDM, so that users can intuitively get the glucose information.

Therefore, there is an urgent need for a management device that is convenient for users to get glucose information in the prior art.

### BRIEF SUMMARY OF THE INVENTION

The invention discloses a management device and method for displaying glucose information. The screen of the management device is divided into the plurality of display areas, comprising at least a first display area for displaying the status of the management device, a second display area for displaying real-time glucose information, and a third display area for displaying historical glucose concentration information. The user can intuitively and easily get the glucose information.

The invention discloses a management device for displaying glucose information, which comprises a screen capable of user interaction. The screen is divided into the plurality of display areas, at least comprising a first display area for displaying the status of the management device, a second display area for displaying real-time glucose information, and a third display area for displaying historical glucose concentration information, wherein at least one display area further comprises calibration information. A power button is at least used to activate or deactivate the screen. And a home key, which is at least used to return the interface displayed on the screen to the home screen.

According to one aspect of the invention, the first display area comprises at least a power icon, a time icon and a prompt mode icon.

According to one aspect of the invention, the power icon, the time icon and the prompt mode icon have different display forms under different operation or setting states.

According to one aspect of the invention, the second display area at least comprises a glucose tendency icon, a glucose concentration value, a glucose concentration detection time and a closed circle, and a tendency of glucose, the glucose concentration value and the glucose concentration detection time are located in the closed circle.

According to one aspect of the invention, the tendency of glucose is represented by a trend icon.

According to one aspect of the invention, the trend icon is filled with the suggestive color.

According to one aspect of the invention, the tendency of glucose is represented by coded color.

According to one aspect of the invention, the color is filled in the background area of the closed circle or the second display area.

According to one aspect of the invention, when an event is triggered, at least one of the tendency icon, the glucose concentration value, and the glucose concentration detection time is replaced by an event icon.

According to one aspect of the invention, the event at least comprises a warm-up of glucose probe, a glucose probe calibration, a loss of signal, an abnormal glucose probe detection or an abnormal glucose concentration value.

According to one aspect of the invention, the third display area comprises a dynamic graph with time as the horizontal axis and glucose concentration value as the vertical axis, for displaying historical glucose information.

According to one aspect of the invention, the real-time glucose concentration value is located at the right side of the dynamic graph.

According to one aspect of the invention, when the user taps the dynamic graph, the information display associated with the historical glucose information is expanded in the dynamic graph.

According to one aspect of the invention, the second display area and the third display area update display at fixed time intervals.

According to one aspect of the invention, the fixed time interval is 2 min.

According to one aspect of the invention, when an alarm event is triggered, an alarm interface replaces at least one of the display areas of the home screen.

The invention also discloses a method of displaying glucose information on the screen of the management device, which receives glucose information from a continuous glucose detection device. Displaying status information of the management device in a first display area of the screen. Displaying real-time glucose information in a second display area of the screen. Displaying historical glucose information in a third display area of the screen. Wherein the third display area is configured to expand the information display associated with the historical glucose information in response to an instruction input of the user.

According to one aspect of the invention, the third display area comprises a dynamic graph with time as the horizontal axis and glucose concentration value as the vertical axis.

According to one aspect of the invention, the user's instruction input at least comprises taping the dynamic graph.

According to one aspect of the invention, the user's instruction input also comprises the duration and/or times of taping the dynamic graph.

According to one aspect of the invention, the expanded information display of the third display area comprises one or more of a recording time, a historical detection time, a corresponding glucose concentration, and a detection time adjustment icon.

According to one aspect of the invention, tap or move the detection time adjustment icon to adjust the historical detection time.

According to one aspect of the invention, the second display area is configured to replace at least a part of the real-time glucose information with an event icon when an event is triggered.

According to one aspect of the invention, the real-time glucose information shall at least comprise a glucose trend, a glucose concentration value and a glucose concentration detection time.

According to one aspect of the invention, it also comprises a closed circle, and the glucose trend, glucose concentration value and glucose concentration detection time are located in the closed circle.

According to one aspect of the invention, when an alarm is triggered, an alarm interface replaces at least one display area.

The invention also discloses a method of blood glucose information display and alarm, which comprises a screen capable of user interaction. The screen is divided into the plurality of display areas, comprising at least a first display area displaying the status of the management device, a second display area displaying real-time glucose information, and a third display area displaying historical glucose concentration information. A power button is at least used to activate or deactivate the screen. And a home key, which is at least used to return the interface displayed on the screen to the home screen. When an alarm prompt is triggered, an alarm interface replaces at least one of the plurality of display areas, and the alarm interface comprises information corresponding to the alarm prompt.

According to one aspect of the invention, when the alarm prompt is triggered, at least one display area displays an interactive area, and the user inputs a confirmation indication through the interactive area.

According to one aspect of the invention, when the alarm prompt is triggered, the management device prompts the user by a sound output, a vibration output or a light output.

According to one aspect of the invention, the sound output, the vibration output, the light output or the combination of the outputs are adjustable.

According to one aspect of the invention, when the screen is off, the alarm prompt is triggered, the screen is started and enters a lock screen interface, the lock screen interface comprises an unlocking interaction area and an alarm prompt area, and the alarm prompt area comprises information corresponding to the alarm prompt.

According to one aspect of the invention, based on the unlock interaction, the user unlocks the screen, and the screen enters an alarm interface.

According to one aspect of the invention, the alarm prompt comprises alarm prompt with high priority and alert prompt with low priority.

According to one aspect of the invention, when the alarm prompt is triggered, the user inputs the confirmation instruction, and implements the response corresponding to the alarm prompt, the alarm prompt is cleared, otherwise, the alarm prompt is not cleared.

According to one aspect of the invention, when the alert prompt is triggered, the user inputs the confirmation instruction, or presses the power button or the home key, and the warning prompt is cleared.

According to one aspect of the invention, when the alarm prompt is triggered, at least one display area displays an alarm icon corresponding to the alarm prompt.

The invention also discloses a user interaction management device for displaying glucose information, which comprises a touch-sensitive screen capable of user interaction. The home screen of the screen is divided into the plurality of display areas, comprising at least a first display area displaying the status of the management device, a second display area displaying real-time glucose information, and a third display area displaying historical glucose concentration information. A power button is at least used to activate or deactivate the screen. And a home key, which is at least used to return the interface displayed on the screen from a secondary interface to the home screen. Wherein, based on the implementation of user interaction, the screen switches between the home screen and the secondary interface.

According to one aspect of the invention, the implementation of user interaction at least comprises sliding on the screen.

According to one aspect of the invention, the user slides horizontally or vertically on the home screen to enter the secondary interface.

According to one aspect of the invention, the user slides horizontally or vertically on the secondary interface to return to the home screen.

According to one aspect of the invention, the sliding direction of entering the secondary interface is opposite to that of returning to the home screen.

According to one aspect of the invention, the user slides along the diagonal of the screen on the secondary interface to return to the home screen.

According to one aspect of the invention, the home key shall be at least one of a physical button or a touch-sensitive button.

According to one aspect of the invention, the touch-sensitive button is located on the screen.

According to one aspect of the invention, the secondary interface shall at least comprise a notification secondary interface, a shortcut secondary interface, a status secondary interface and a menu secondary interface.

According to one aspect of the invention, the secondary interface also comprises a return soft key, which is used to return to the previous interface of the secondary interface.

According to one aspect of the invention, the secondary interface further comprises a scroll bar and/or a scroll arrow.

Compared with the prior art, the technical scheme of the invention has the following advantages:

The invention discloses a management device for displaying glucose information. The screen of the management device is divided into the plurality of display areas, comprising at least a first display area for displaying the status of the management device, a second display area for displaying real-time glucose information, and a third display area for displaying historical glucose concentration information. At least one display area further comprises sensor calibration information, and the user can intuitively and conveniently get the glucose information in the body and improve the user experience.

Further, the tendency of glucose, glucose concentration value and glucose concentration detection time are located in the closed circle. The glucose information is concentrated in the closed area, and multiple information will not appear scattered. The display content of the screen is more beautiful, which is convenient for the user to get the glucose information and improves the user experience.

Further, according to the duration of the user taping the dynamic graph, the dynamic graph can be expanded and displayed differently, effectively utilizing the display area of the screen, facilitating user operation and improving user experience.

Further, when the alarm event is triggered, the alarm interface replaces at least one of the display areas, and the alarm interface displays information corresponding to the alarm. The user can intuitively and conveniently understand the alarm information through the management equipment, which improves the user experience.

Further, when the user operates interactively on the screen, the screen can be switched between the home screen and the secondary interface. The user can intuitively understand the blood sugar information through the management device, and can conveniently switch to other interfaces to query information and make personalized settings, which improves the user experience.

Further, sliding horizontally or vertically on the home screen of the screen to enter different secondary interfaces, which is convenient to operate and improves the user experience.

### BRIEF DESCRIPTION OF THE DRAWINGS

FIG. 1 is the schematic diagram of the continuous glucose monitoring system according to the embodiment of the invention.

FIG. 2a is the schematic diagram of the home screen according to the embodiment of the invention.

FIG. 2b is the schematic diagram of electric quantity according to the embodiment of the invention.

FIG. 2c is the schematic diagram of the prompt mode according to the embodiment of the invention.

FIG. 2d is the schematic diagram of tendency of glucose according to the embodiment of the invention.

FIG. 2e is the schematic diagram of historical glucose information according to the embodiment of the invention.

FIG. 2f is the schematic diagram of the dynamic graph expansion display step according to the embodiment of the invention.

FIG. 2g is the diagram of the second display area under the special condition according to the embodiment of the invention.

FIG. 2h is the schematic diagram of Warm-up of the probe according to the embodiment of the invention.

FIG. 3a is the schematic diagram of the lock screen according to the embodiment of the invention.

FIG. 3b is the schematic diagram of the alarm prompt when the screen is locked according to the embodiment of the invention.

FIG. 3c is the schematic diagram of the alarm prompt after the screen is unlocked according to the embodiment of the invention.

FIG. 4a is the schematic diagram of the notification secondary interface according to the embodiment of the invention.

FIG. 4b is the schematic diagram of the shortcut secondary interface according to the embodiment of the invention.

FIG. 4c is the schematic diagram of the status secondary interface according to the embodiment of the invention.

FIG. 4d is the schematic diagram of another state information interface according to the embodiment of the invention.

FIG. 4e is the schematic diagram of the device information interface according to the embodiment of the invention.

FIG. 4f is the schematic diagram of the menu interface according to the embodiment of the invention.

FIGS. 5a to 5c are schematic diagrams of the operation mode of returning to the home screen according to the embodiment of the invention.

FIGS. 6a to 6d are schematic diagrams of display contents of the second display area according to the embodiment of the invention.

FIG. 7 is the schematic diagram of the plurality of quantity indicator lights according to the embodiment of the invention.

FIG. 8 is the schematic diagram of the scroll bar according to the embodiment of the invention.

FIG. 9a is the schematic diagram of the alarm prompt according to the embodiment of the invention.

FIG. 9b is the schematic diagram of the alert prompt according to the embodiment of the invention.

FIG. 9c is the flow diagram of clearing the alarm prompt according to the embodiment of the invention.

### DETAILED DESCRIPTION

As mentioned above, with the improvement of the functions of PDM, the contents and interfaces to be displayed on the screen gradually increase. How to reasonably arrange the contents and interfaces displayed on the screen also needs to be considered in the design process of PDM, so that users can intuitively get the glucose information.

In order to solve this problem, the invention provides a management device for displaying glucose information. The screen of the management device is divided into the plurality of display areas, comprising at least a first display area displaying the status of the management device, a second display area displaying real-time glucose information, and a third display area displaying historical glucose concentration information, wherein at least one display area further comprises sensor calibration information, users can intuitively and conveniently get the glucose information in the body through the home screen of the management device.

Various exemplary embodiments of the invention will now be described in detail with reference to the attached drawings. It is understood that, unless otherwise specified, the relative arrangement of parts and steps, numerical expressions and values described in these embodiments shall not be construed as limitations on the scope of the invention.

In addition, it should be understood that the dimensions of the various components shown in the attached drawings are not necessarily drawn to actual proportions for ease of description, e. g. the thickness, width, length or distance of some elements may be enlarged relative to other structures.

The following descriptions of exemplary embodiments are illustrative only and do not in any sense limit the invention, its application or use. Techniques, methods and devices known to ordinary technicians in the relevant field may not be discussed in detail here, but to the extent applicable, they shall be considered as part of this manual.

It should be noted that similar labels and letters indicate similar items in the appending drawings below, so that once the item is defined or described in one of the appending drawings, there is no need to discuss it further in the subsequent appending drawings.

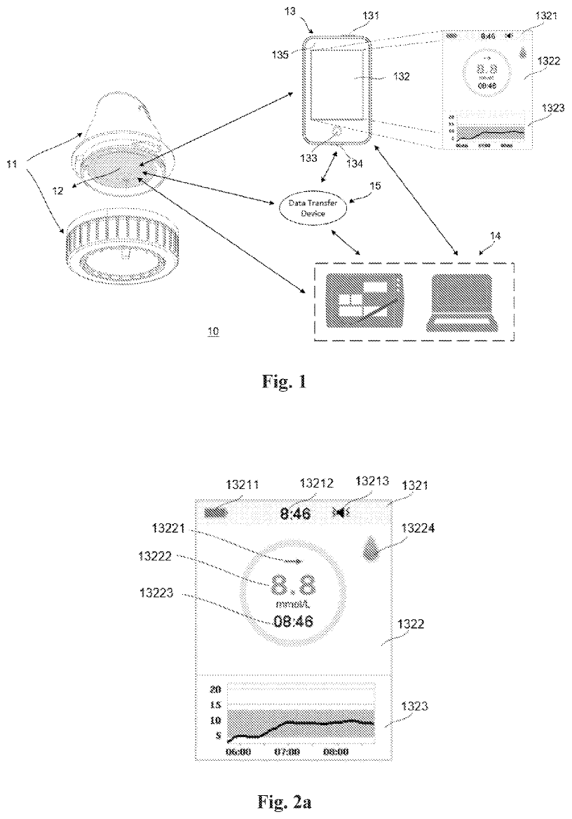

FIG. 1 is the schematic diagram of the continuous glucose monitoring system according to the embodiment of the invention.

Referring to FIG. 1, in some embodiments of the invention, the continuous glucose detection system 10 comprises the auxiliary installer 11, the continuous glucose detection device 12, and the personal diabetes management device (hereinafter referred to as the management device) 13. The auxiliary installer 11 is used to install the continuous glucose detection device 12 on the surface of the user's skin. After the continuous glucose detection device 12 is installed on the surface of the user's skin, the continuous glucose detection device 12 acquires the glucose concentration information in the user's body and sends the glucose concentration information to the management device 13. After receiving the glucose concentration information, the management device 13 analyzes and collates it, and finally displays it on the screen 132 of the management device 13 in the intuitive manner.

In other embodiments of the invention, the continuous glucose detection system 10 further comprises the diabetes secondary management device (hereinafter referred to as the secondary management device) 14. The secondary management device 14 may be the computer, the mobile phone, and etc. Their user may be the user himself or someone other than the user, for example, the guardian or the doctor of the user. In some embodiments of the invention, the content displayed by the secondary management device 14 is consistent with the content displayed by the management device 13.

In some embodiments of the invention, the continuous glucose detection device 12 is wirelessly connected to the management device 13 (or the secondary management device 14) through Bluetooth, Wi-Fi, or the Internet. In the preferred embodiment of the invention, the continuous glucose detection device 12 is wirelessly connected to the management device 13 through Bluetooth, which can save the power consumption of the continuous glucose detection device 12 and extend its service life.

In some embodiments of the invention, the effective communication distance between the continuous glucose detection device 12 and the management device 13 is 0-20 m. when the actual distance between the continuous glucose detection device 12 and the management device 13 exceeds the effective communication distance, the management device 13 will not be able to receive or fully receive the signals transmitted by the continuous glucose detection device 12, so the glucose information cannot be displayed on the screen 132.

In some embodiments of the invention, the continuous glucose detection device 12 can adjust the effective communication distance by adjusting the power of signal, instead of using the maximum power for the long time to maintain the maximum effective communication distance, so as to further save the power consumption of the continuous glucose detection device 12 and extend its service life.

In some embodiments of the invention, the management device 13 may establish the wireless communication connection with the secondary management device 14 through Bluetooth, Wi-Fi or the Internet, or establish the wired communication connection with the secondary management device 14 through USB, serial port, Ethernet, etc., to realize data transmission between the management device 13 and the secondary management device 14.

In some embodiments of the invention, the continuous glucose detection system 10 further comprises the data transfer device 15. The data transfer device 15 can be placed within the effective communication distance of the continuous glucose detection device 12, and establish the wireless Bluetooth communication connection with the continuous glucose detection device 12, and establish the wireless communication connection with the management device 13 (or the secondary management device 14). Since the data transfer device 15 can use additional power, the wireless communication connection with the longer signal transmission distance such as Wi-Fi or the Internet can be established between the data transfer device 15 and the management device 13 (or the secondary management device 14). For example, the effective communication distance between the data transfer device 15 and the management device 13 can be 50 m, 200 m, even 1000 m, or more. In the embodiment of the invention, after receiving the glucose information transmitted by the continuous glucose detection device 12, the data transfer device 15 transfers the glucose information to the management device 13, so as to realize ultra-long-distance information transmission.

In some embodiments of the invention, the management device 13 comprises the power button 131, the screen 132, the home key 133, the charging port 134, the indicator light 135, and the processor (not shown). The power button 131 is used to activate or deactivate the screen 132, the screen 132 is used to display glucose information, the home key 133 is used to return the display interface of the screen 132 to the home screen, the charging port 134 is used to connect the charging line and charge the management device 13, and the indicator light 135 is used to flash according to the required prompt information according to the predetermined logic.

### Home Screen

In some embodiments of the invention, the screen 132 comprises three display areas, namely, the first display area 1321 for displaying the status of the management device, the second display area 1322 for displaying real-time glucose information, and the third display area 1323 for displaying historical glucose concentration information.

In other embodiments of the invention, the screen 132 may also comprise other display areas for displaying additional available information, such as insulin infusion information, exercise information, mobile phone number, geodynamic graphic location information, user physiological information, etc. In other embodiments of the invention, the above information may also be displayed in one or more of the first display area 1321, the second display area 1322, or the third display area 1323.

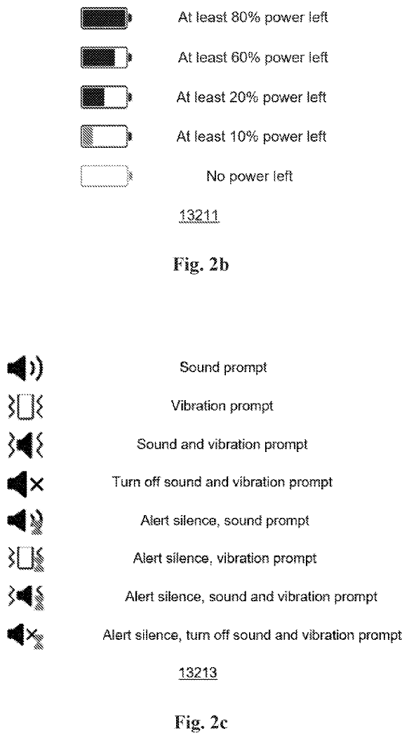

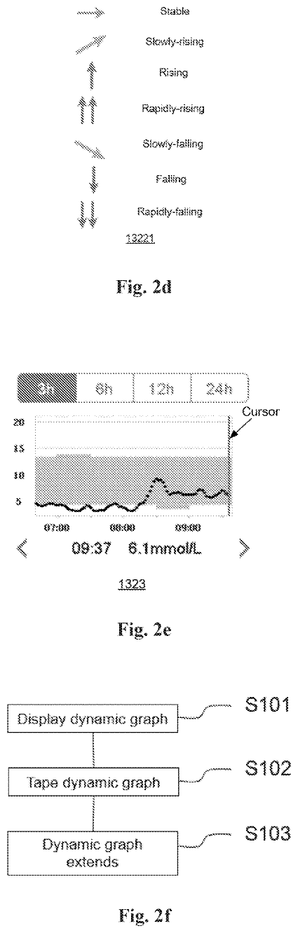

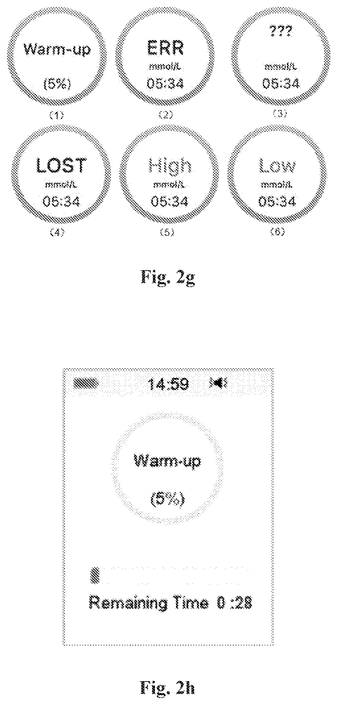

FIG. 2a is the schematic diagram of the home screen according to the embodiment of the invention. FIG. 2b is the schematic diagram of electric quantity according to the embodiment of the invention. FIG. 2c is the schematic diagram of the prompt mode according to the embodiment of the invention. FIG. 2d is the schematic diagram of tendency of glucose in the embodiment of the invention. FIG. 2e is the schematic diagram of historical glucose information according to the embodiment of the invention. FIG. 2f is the schematic diagram of the dynamic graph expansion display step according to the embodiment of the invention. FIG. 2g is the display diagram of the second display area of the embodiment of the invention under special conditions. FIG. 2h is the schematic diagram of Warm-up of the glucose probe according to the embodiment of the invention.

Referring to FIG. 2a, in some embodiments of the invention, the first display area 1321 is used to display the status of the management device 13, such as the power icon 13211, the time icon 13212, and the prompt mode icon 13213.

Referring to FIG. 2b, in some embodiments of the invention, the power icon 13211 represents the power left of the management device 13 in the form of the icon filling percentage. For example, when the power icon 13211 is filled with 100%, the management device 13 has at least 80% of the power left. When the power icon 13211 is filled with 80%, at least 60% of the power remains in the management device 13. When the power icon 13211 is filled with 40%, at least 20% of the power remains in the management device 13. When the power icon 13211 is filled with 20%, at least 10% of the power remains in the management device 13. When the power icon 13211 is filled with 0, the management device 13 has no power left. The above percentages are only illustrative, and other filling percentages may be used to express the power left of the management device 13. In the preferred embodiment of the invention, when the power left of the management device 13 is less than 20%, the color of the power icon 13211 is changed to the suggestive color, for example, red. In another preferred embodiment of the invention, when the management device 13 consumes power and charges, the colors of the power icon 13211 are different to distinguish.

Referring back to FIG. 2a, in some embodiments of the invention, the time icon 13212 is used to display the local time and can be adjusted in the setting interface, such as 24-hour format or 12-hour format.

Referring to FIG. 2c, in some embodiments of the invention, the prompt mode icon 13213 may have different illustrations according to the user's selection of sound or vibration prompts. For example, according to FIG. 2c, the prompt mode icons 13213 are sound prompt, vibration prompt, sound and vibration prompt, turn off sound and vibration prompt, sensor alarm silence simultaneous sound prompt, sensor alarm silence simultaneous vibration prompt, sensor alarm silence simultaneous sound and vibration prompt, and sensor alarm silence simultaneous sound and vibration prompt from top to bottom. Here, sensor alarm silence means that when the glucose concentration value exceeds the high/low threshold, no alarm prompt appears on the interface of the management device 13. The alarm prompt on the interface of the management device 13 will be described in detail below.

Referring back to FIG. 2a, in some embodiments of the invention, the second display area 1322 is used to display glucose information, for example, the glucose tendency icon 13221, the glucose concentration value 13222, the glucose concentration detection time 13223, and the glucose probe calibration icon 13224.

Referring to FIG. 2d, in some embodiments of the invention, the glucose tendency icon 13221 is used to express the rising or falling trend of glucose. In the embodiment of the invention, the glucose tendency icon 13221 is expressed in the form of the trend arrow. The direction and inclination of the trend arrow can inform the user whether the glucose concentration in the body is rising or falling, and the speed of the rise or fall. For example, the correspondence between the trend arrow and the tendency of glucose may be as follows:

When the arrow is horizontal, it indicates that the current glucose concentration value remains stable, and the fluctuation range of increase or decrease is small.

When the arrow is inclined upward with respect to the horizontal plane, it indicates that the current glucose concentration value is slowly rising.

When the arrow is upward vertical to the horizontal plane, it indicates that the current glucose concentration value is rising.

When the arrows are upward vertical to the horizontal plane, it indicates that the current glucose concentration value is rapidly rising.

The horizontal mirror direction of the arrow indicates that the current glucose concentration value is falling to the corresponding degree.

In some embodiments of the invention, the rates of “slowly rising”, “slowly falling”, “rising”, “falling”, “rapidly rising” and “rapidly falling” of glucose concentration values can be set by the user or his supervisor/medical staff, for example, the rate threshold value of rise/fall is set to 0.110 mmol/L/min, and the rate threshold value of rapid rise/rapid fall is set to 0.170 mmol/L/min, below the rise/fall rate threshold is slowly rising/slowly falling. In other embodiments of the invention, the rate threshold of rise/fall of glucose concentration value can be set as different values according to the needs of users or their supervisors/medical personnel.

In the preferred embodiment of the invention, filling the arrows with suggestive colors can better remind the user of the current tendency of glucose, for example, filling the horizontal arrows with green, filling the inclined arrows with yellow, and filling the vertical arrows with red. It should be understood by those skilled in the art that other icons or colors can also be used for the expression of tendency of glucose, and there is no limitation here.

Referring back to FIG. 2a, in some embodiments of the invention, the glucose concentration value 13222 is used to represent the glucose concentration detected by the latest glucose probe, and the glucose concentration detection time 13223 is used to represent the detection time of the latest glucose probe. The glucose probe detects the glucose concentration of the user at fixed time intervals, and the second display area 1322 displays only the glucose concentration detected most recently. In the preferred embodiment of the invention, the glucose probe detects the glucose concentration of the user at the time interval of 2 min, that is, the second display area 1322 also refreshes the glucose concentration value 13222 and the glucose concentration detection time 13223 at the time interval of 2 min.

In some embodiments of the invention, the unit of glucose concentration value 13222 is mmol/L. In other embodiments of the invention, the unit of glucose concentration value 13222 is mg/dl.

In the preferred embodiment of the invention, the suggestive color may be filled for the glucose concentration value 13222. For example, when the glucose concentration value 13222 is between the hyperglycemia/hypoglycemia thresholds, the glucose concentration value 13222 is filled with green. When the glucose concentration value 13222 is higher than the hyperglycemia threshold, the glucose concentration value 13222 is filled with yellow. When the glucose concentration value 13222 is lower than the hypoglycemic threshold, the glucose concentration value 13222 is filled with red.

Referring back to FIG. 2a, in some embodiments of the invention, the glucose probe calibration icon 13224 is used to record the calibration time of the glucose probe and display it to the user in the form of countdown. The form of countdown can be set according to the user's preference. In the embodiment of the invention, the glucose probe calibration icon 13224 is the water drop diagram. After the glucose probe calibration is completed, the water drop diagram is filled with 100% color. With the extension of time, the color filling degree of the water drop diagram gradually decreases until there is no color filling. At this time, it is necessary to calibrate the glucose probe again. When the water droplet diagram is not filled with color, the management device 13 emits sound or vibration to the user to prompt the user. The above water drop diagram is only for reference.

In other embodiments of the invention, the glucose probe does not need to be calibrated during use, so the glucose probe calibration icon 13224 does not need to be displayed on the screen 132.

Referring back to FIG. 2a, in some embodiments of the invention, the third display area 1323 is used to display historical glucose concentration information. The third display area 1323 comprises the dynamic graph whose vertical axis Y is the glucose concentration scale and the horizontal axis X is the time scale.

In some embodiments of the invention, the unit of the vertical axis of the dynamic graph is mmol/L, and there are four scales on it, which are 5, 10, 15 and 20, respectively, corresponding to 90, 180, 270 and 360 (mg/dl). In the embodiment of the invention, two protruding scales are also provided on the vertical axis, namely, the hypoglycemic scale and the hyperglycemic scale. For example, they can be set to 4.4 mmol/L (80 mg/dl) and 13.3 mmol/L (240 mg/dl). In the preferred embodiment of the invention, the above two prominent scales are filled with the distinguishing color, such as green, to indicate that the interval is normal glucose concentration.

In other embodiments of the invention, the hypoglycemic scale and the hyperglycemic scale can be modified and set by the user or his/her supervisor according to needs. The above hypoglycemic scale and the hyperglycemic scale are only illustrative.

In some embodiments of the invention, the horizontal axis of the dynamic graph has three time-values corresponding to three periods, that is, the dynamic graph can display the glucose concentration information in the past three hours. In other embodiments of the invention, the horizontal axis of the dynamic graph may have two time-values, four time-values, five time-values, etc., which are not limited here.

Referring to FIGS. 2e and 2f, in some embodiments of the invention, taping the dynamic graph can enter the interface shown in FIG. 2e, where the recording time of glucose information can be selected, that is, the aforementioned time value.

In some embodiments of the invention, in step S101, the third display area 1323 displays the dynamic graph as described above, in step S102, the user tapes the dynamic graph to expand the information display, and in step S103, the dynamic graph is displayed based on the user's tap to expand the information display.

In some embodiments of the invention, the dynamic graph may expand different information displays according to the duration and/or the times the user taps the dynamic graph. For example, when the user briefly taps the dynamic graph, the adjustment icon like movable vertical cursor is expanded and displayed on the dynamic graph. When the user taps the dynamic graph for the period of time, for example, the user taps the dynamic graph for Is, the adjustment icon like arrow “<” (“>”) will be expanded and displayed on the dynamic graph. When the user taps the dynamic graph twice in the short time, the recording duration of the extended display on the dynamic graph. When the user taps the dynamic graph three times in the short time, the historical detection time and the corresponding glucose concentration are expanded and displayed on the dynamic graph. This is only the exemplary description and is not limited in detail.

In some embodiments of the invention, the user adjusts the historical detection time by moving the vertical cursor or clicking the arrow “<” (“>”) to query the glucose concentration value detected at the certain time in the history.

In some embodiments of the invention, since the glucose concentration value is recorded at the frequency of 2 min, the time jump interval on the interface is 2 min when moving the vertical cursor or clicking the arrow.

In some embodiments of the invention, historical glucose information is displayed near the dynamic graph, for example, below the dynamic graph, and displayed in the form of (time glucose concentration value).

In some embodiments of the invention, when the vertical cursor is moved or the arrow is clicked, the historical glucose information can also be directly displayed in the dynamic graph or displayed in other areas, and its specific display form can also be changed, which is not limited here.

In some embodiments of the invention, the dynamic graph shown in FIG. 2a or FIG. 2e is dynamic. Since the glucose probe detects the glucose concentration every 2 minutes and sends the glucose concentration information to the management device 13, the management device 13 needs to update the glucose concentration information every 2 minutes and update the most recently detected glucose concentration on the right side of the dynamic graph, the glucose concentration detected in the early stage moves to the left by one detection interval unit in the detection sequence, i.e. 2 min.

In some embodiments of the invention, the glucose concentration values in the dynamic graph are discrete points, that is, one detection time corresponds to one glucose concentration value, and the glucose concentration values corresponding to different detection times are independent. In other embodiments of the invention, the glucose concentration values in the dynamic graph are smoothly connected by curves through algorithms, that is, one detection time corresponds to one glucose concentration value, and the glucose concentration values corresponding to different detection times are connected by smooth curves. In still other embodiments of the invention, the glucose concentration values in the dynamic graph are connected by broken lines, that is, one detection time corresponds to one glucose concentration value, and the glucose concentration values corresponding to two adjacent detection times are connected by straight lines.

### Special Condition

Referring back to FIG. 2a, under normal conditions, the second display area 1322 displays information such as the glucose concentration value 13222 as described above. Under some special conditions, the second display area 1322 will display other information and at least partially replace the aforementioned information such as the glucose concentration value 13222.

In some embodiments of the invention, when a special condition is triggered, part of the display information in the second display area 1322 is replaced by event icon.

Referring to FIG. 2g and FIG. 2h, in some embodiments of the invention, under the special condition (1), when the glucose probe just penetrates the user's skin and activates to work, there is the period of warm-up time. At this time, the content displayed in the second display area 1322 is “warm-up” and the percentage of warm-up. After the warm-up is completed, the second display area 1322 is changed to the content shown in FIG. 2a.

In the preferred embodiment of the invention, during the heating process of the glucose probe, the progress bar of the warm-up is displayed on the screen, and the progress of the progress bar is consistent with the completion percentage of the warm-up. In another preferred embodiment of the invention, the screen also displays the countdown of the completion of the warm-up. Generally, the time required for the warm-up of the glucose probe is 30 min.

In some embodiments of the invention, under the special condition (2), when the glucose probe needs to be recalibrated, the content displayed in the second display area 1322 is “ERR”, indicating that the glucose probe needs to be recalibrated after 15 min. After the recalibration is completed, the second display area 1322 is changed to the content shown in FIG. 2a.

In some embodiments of the invention, under the special condition (3), when the management device 13 and the continuous glucose detection device 12 establish the communication connection, if the continuous glucose detection device 12 is unable to detect the glucose due to the displacement of the glucose probe under the skin, or the electronic circuit of the continuous glucose detection device 12 fails, and the glucose probe has no current, the continuous glucose detection device 12 cannot send the glucose concentration information to the management device 13, the content displayed in the second display area 1322 is “???”. The second display area 1322 is changed to the content shown in FIG. 2a after the user removes the fault and the continuous glucose detection device 12 can send the glucose concentration information to the management device 13.

In some embodiments of the invention, under the special condition (4), if the distance between the management device 13 and the continuous glucose detection device 12 exceeds the signal effective distance, or the signal of the continuous glucose detection device 12 is shielded, and the management device 13 cannot receive the signal of the continuous glucose detection device 12 for more than the period of time, the content displayed in the second display area 1322 is “LOST”, when the management device 13 receives the signal from the continuous glucose detection device 12 again, the second display area 1322 is changed to the content shown in FIG. 2a. In the embodiment of the invention, the signal loss time is set to 10 min, which is only exemplary.

In some embodiments of the invention, under the special condition (5) or (6), there are upper and lower limits on the glucose concentration value that can be displayed by the management device 13, for example, the lowest displayable glucose concentration value is 2.2 mmol/L (40 mg/dl), and the highest displayable glucose concentration value is 22.2 mmol/L (400 mg/dl). When the actual glucose concentration detected by the glucose probe is higher than the highest displayable glucose concentration value or lower than the lowest displayable glucose concentration value, the content displayed in the second display area 1322 is “High” or “Low”, indicating that the current glucose concentration value is abnormal. When the glucose concentration value returns to the range of the highest displayable glucose concentration value and the lowest displayable glucose concentration value, the second display area 1322 is changed to the content shown in FIG. 2a.

### Screen Locking and Unlocking

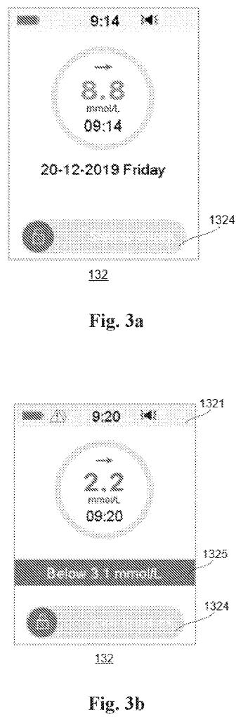

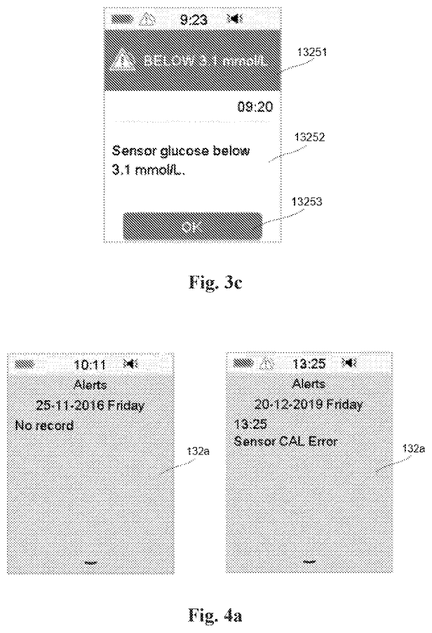

FIG. 3a is the schematic diagram of the locked screen according to the embodiment of the invention. FIG. 3b is the schematic diagram of the alarm prompt when the screen is locked according to the embodiment of the invention. FIG. 3c is the schematic diagram of alarm prompt after unlocking the screen according to the embodiment of the invention.

In some embodiments of the invention, the screen 132 is not taped for the long time or enters the sleep mode after the user presses the power button 131. The screen 132 does not display any content and the screen backlight is turned off. When the user presses the power button 131 again, the screen backlight is illuminated, the screen 132 enters the wake-up mode and displays the screen locked interface as shown in FIG. 3a. The user needs to slide the slider 1324 to unlock the screen 132, enter the home screen as shown in FIG. 2a.

Referring to FIG. 3b, in some embodiments of the invention, in the sleep mode, when the management device 13 triggers the hypoglycemia or hyperglycemia alarm, the screen 132 can also enter the wake-up mode. At this time, the wake-up screen 132 adds the hypoglycemia or hyperglycemia alarm prompt area 1325, and reminds the user in the form of text, and displays “ [symbol] ” icon in the first display area 1321. In the preferred embodiment of the invention, the alarm prompt area 1324 may be filled with the prompt color, such as red or orange.

Referring to FIG. 3c, in some embodiments of the invention, after the user unlocks the screen 132 by sliding the slider 1324 under the alarm prompt, the screen 132 switches to display the interface shown in FIG. 3c, and the alarm prompt area 1325 is changed to the first alarm area 13251, the second alarm area 13252, and the confirmation area 13253. The first alarm area 13251 and the second alarm area 13252 both display the alarm content in text, the first alarm area 13251 or the second alarm area 13252 or the first alarm area 13251 and the second alarm area 13252 are filled with the suggestive color, such as red or orange, to enhance the alarm prompt effect. After the user clicks the “OK” key in the confirmation area 13253, the management device 13 determines that the user has understood the alarm prompt, and the screen 132 returns to the home screen as shown in FIG. 2a, while the icon “A” in the first display area 1321 disappears, and as described above, when the hyperglycemia alarm is triggered, the glucose concentration value 13222 is filled with yellow. When the hypoglycemia alarm is triggered, the glucose concentration value 13222 is filled with red.

Referring to FIG. 1, in some embodiments of the invention, when the hyperglycemia/hypoglycemia alarm prompt is triggered, or under special conditions, the indicator light 135 flashes according to the prompt type.

### Secondary Interface

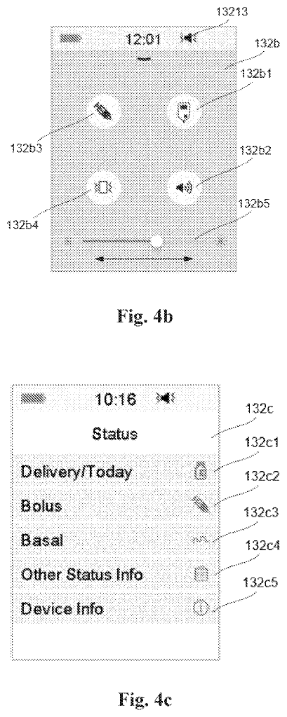

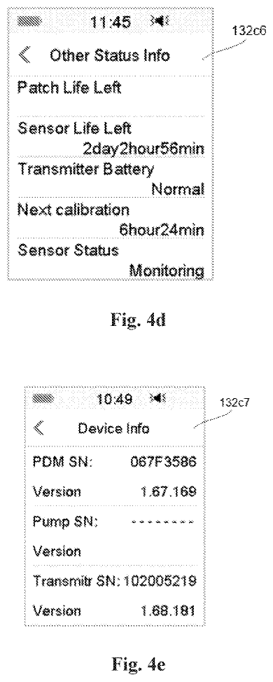

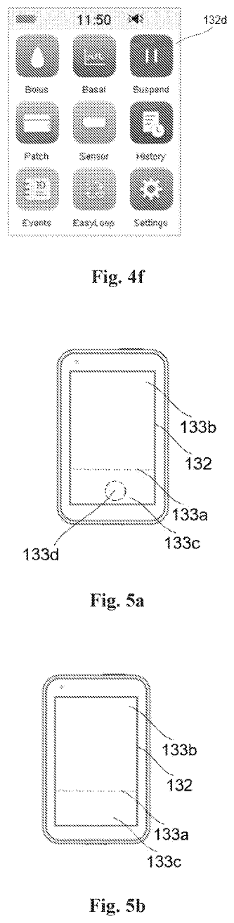

FIG. 4a is the schematic diagram of the notification secondary interface according to the embodiment of the invention. FIG. 4b is the schematic diagram of the shortcut secondary interface according to the embodiment of the invention. FIG. 4c is the schematic diagram of the status secondary interface according to the embodiment of the invention. FIG. 4d is the schematic diagram of another state information interface according to the embodiment of the invention. FIG. 4e is the schematic diagram of the device information interface according to the embodiment of the invention. FIG. 4f is the schematic diagram of the menu secondary interface according to the embodiment of the invention.

In some embodiments of the invention, some secondary interfaces can be accessed by sliding on the home screen. Here, “sliding” can be sliding from top to bottom, from bottom to top, from left to right or from right to left. Different secondary interfaces can be accessed according to different sliding actions.

Referring to FIG. 4a, in some embodiments of the invention, the home screen slides from top to bottom to enter the notification secondary interface 132a. The notification secondary interface 132a is used to display the alarm prompt and occurrence time of the current day, such as hyperglycemia/hypoglycemia alarm, signal loss, glucose probe calibration, etc. if there is no alarm prompt on the current day, then “no record” will be displayed on the notification secondary interface 132a. If there are multiple alarm prompts on the current day, the latest alarm will be displayed on the top of the notification secondary interface 132a, and sequentially displayed on the notification secondary interface 132a according to the time sequence of occurrence of the alarm prompt.

In some embodiments of the invention, when the notification secondary interface 132a is slid from bottom to top, or the home key 133 is taped, the user can return to the home screen.

Referring to FIG. 4b, in some embodiments of the invention, the home screen slides from bottom to top to enter the shortcut secondary interface 132b. The shortcut secondary interface 132b comprises the calibration icon 132b1, the sound icon 132b2, the bolus infusion icon 132b3, the vibration icon 132b4, and the background light icon 132b5.

In some embodiments of the invention, click the calibration icon 132b1 to enter the glucose probe calibration program.

In some embodiments of the invention, clicking the bolus infusion icon 132b3 can enter the bolus insulin infusion program. The premise of using the above procedure is that the management device 13 has established the communication connection with the insulin pump.

In some embodiments of the invention, clicking the sound icon 132b2 and/or the vibration icon 132b4 can adjust the sound and vibration prompt of the management device 13. For example, clicking the sound icon 13262 can open or close the sound prompt, clicking the vibration icon 132b4 can open or close the vibration prompt, and the prompt mode icon 13213 can adjust and change according to the state of the sound prompt and vibration prompt, see FIG. 2c and its description text for specific adjustment results.

In some embodiments of the invention, pressing the background light icon 132b5 and sliding it left or right can adjust the brightness of the background light of the screen. In the embodiments of the invention, sliding the background light icon 13265 to the left reduces the brightness of the background light, and sliding the background light icon 13265 to the right increases the brightness of the background light. In the embodiment of the invention, the brightness of the background light is set at 10 levels. Here, the way of adjusting the background light brightness is only described for reference. In other embodiments of the invention, the background light brightness adjustment can also be realized by sliding the background light icon 132b5 up and down and other methods, which are not limited here.

In some embodiments of the invention, the shortcut secondary interface 132b can slide from top to bottom, or tap the home key 133 to return to the home screen.

Referring to FIG. 4c, in some embodiments of the invention, the status secondary interface 132c can be accessed by sliding from left to right on the home screen. In the status secondary interface 132c, the infusion column 132cl, the bolus column 132c2, the basal column 132c3, the other status information column 132c4 and the equipment information column 132c5 are displayed in the form of horizontal columns from top to bottom. The user can click each column to enter the corresponding status query interface.

In some embodiments of the invention, the user needs to establish the communication connection between the management device 13 and the insulin pump before he can click the use infusion column 132cl, the bolus column 132c2 and the basic volume column 132c3, and then enter the corresponding status query interface, which will not be described in detail here. In other embodiments of the invention, when the management device 13 does not establish the communication connection with the insulin pump, the status secondary interface 132c does not display the infusion column 132cl, the bolus column 132c2, and the basic volume column 132c3.

Referring to FIG. 4d, in some embodiments of the invention, in the status secondary interface 132c, the user clicks the other state information column 132c4, and the status secondary interface 132c switches to display the other state information interface 132c6 as shown in FIG. 4d. In the other state information interface 132c6, the remaining life of the insulin pump (* day * hour * min), the remaining life of the glucose probe (* day * hour * min), next calibration timing (* hour * min) and glucose probe status. In the embodiment of the invention, the display order of each state information is only described by way of example.

In some embodiments of the invention, in the other status information interface 132c6, click the soft key “<” on the screen to return to the status secondary interface 132c, or tap the home key 133 to return to the home screen.

Referring to FIG. 4e, in some embodiments of the invention, in the status secondary interface 132c, the user clicks the device information column 132c5, and the status secondary interface 132c switches to display the device information interface 132c7 shown in FIG. 4e. In the device information interface 132c7, the device code of the management device 13, the device code of the insulin pump (if the communication connection has been established) and the device code of the continuous glucose monitoring device 12 are displayed in order from top to bottom.

In some embodiments of the invention, in the device information interface 132c7, click the soft key “<” on the screen to return to the status secondary interface 132c, or tap the home key 133 to return to the home screen.

In some embodiments of the invention, the status secondary interface 132c slides from right to left, or the home key 133 is taped to return to the home screen.

Referring to FIG. 4f, in some embodiments of the invention, the home screen slides from right to left to enter the menu interface 132d, where the management device 13 can be queried and set. For example, query historical records, such as calibration records and alarm records. Query events, such as eating events and exercise events. Set hyperglycemia/hypoglycemia threshold, language, date, etc.

In some embodiments of the invention, sliding the menu interface 132d from left to right or taping the home key 133 can return to the home screen.

### Return to the Home Screen

FIGS. 5a to 5c are schematic diagrams of the operation mode of returning to the home screen according to the embodiment of the invention.

In some embodiments of the invention, the home key 133 is the first command button for returning to the home screen. In any interface, the user can return to the main page by taping the home key 133.

Referring back to FIG. 1, in some embodiments of the invention, the home key 133 may be the physical button independent of the screen 132. Separating the screen 132 from the home key 133 can prevent the user from taping the screen 132 by mistake when sliding on the screen 132, thereby affecting the user experience.

Referring to FIG. 5a, in some embodiments of the invention, the screen 132 is divided into two areas by the dividing line 133a, namely, the upper area 133b comprising the first display area 1321, the second display area 1322, and the third display area 1323, and the lower area 133c. In some embodiments of the invention, the lower area 133c comprises the closed area 133d, which is the tap sensitive screen. When the user taps the closed area 133d, the upper area 133b will return to the home screen from any interface. Therefore, the closed area 133d can be used as the home key 133.

Referring to FIG. 5b, in other embodiments of the invention, the lower area 133c does not comprise the closed area 133d. The lower area 133c is the tap sensitive screen as the whole. When the user taps any position of the lower area 133c, the upper area 133b will return to the home screen from any interface. Therefore, the lower area 133c can be used as the home key 133.

Referring to FIG. 5c, in some embodiments of the invention, when sliding diagonally on the screen 132, the screen 132 will return from any interface to the home screen. In some embodiments of the invention, sliding along the diagonal line may be the diagonal line that slides across the entire screen 132. As shown by the solid line arrow in FIG. 5c, the sliding direction may be from bottom to top or from top to bottom. In other embodiments of the invention, the sliding along the diagonal may be the distance over one end, for example, ½ or ⅓ or ¼ of the diagonal. As shown by the dotted arrow in FIG. 5c, the sliding direction may be from bottom to top or from top to bottom.

### Glucose Concentration Information

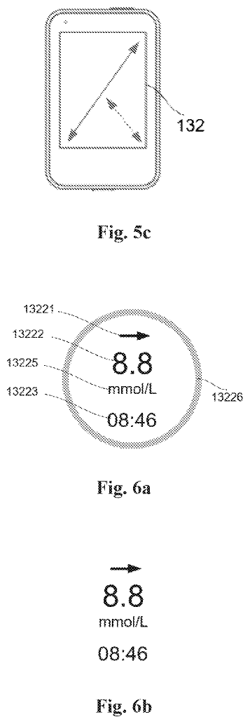

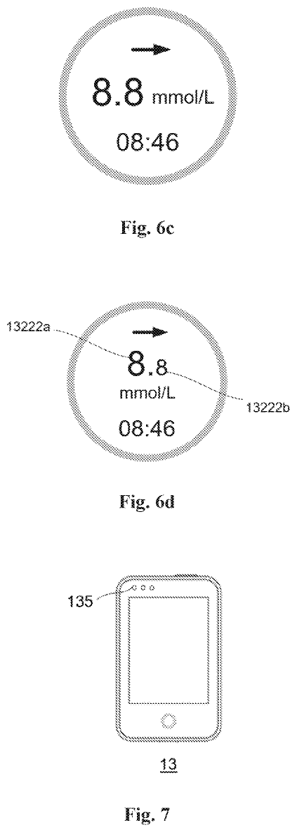

FIGS. 6a to 6d are schematic diagrams of display contents of the second display area according to the embodiment of the invention.

Referring back to FIG. 2a, in some embodiments of the invention, glucose concentration information is displayed in the second display area 1322 of the home screen. The second display area 1322 is located in the middle of the screen 132 to facilitate the user to view the glucose concentration information.

Referring to FIG. 6a, in some embodiments of the invention, the second display area 1322 further comprises the glucose concentration unit 13225 and the closed circle 13226. The closed circle 13226 is used to enclose the glucose tendency icon 13221, the glucose concentration value 13222, the glucose concentration unit 13225, and the glucose concentration detection time 13223 in the middle of the second display area 1322, so that the above contents are polymerized on the screen and appear more beautiful. Without the closed circle 13226, the above contents will appear scattered on the screen, as shown in FIG. 6b.

In some embodiments of the invention, the closed circle 13226 may be circular, square, or polygonal, and is not limited in the invention.

In some embodiments of the invention, the closed circle 13226 may be filled with changeable colors, and the tendency of glucose may be expressed by coded color. For example, the corresponding relationship between the color filling and the tendency of glucose may be as follows:

When the glucose concentration is stable, the closed circle 13226 is filled with green.

When the glucose concentration rises slowly, the closed circle 13226 is filled with light-yellow.

When the glucose concentration rises, the closed circle 13226 is filled with yellow.

When the glucose concentration rises rapidly, the closed circle 13226 is filled with dark-yellow.

When the glucose concentration falls slowly, the closed circle 13226 is filled with light-red.

When the glucose concentration falls, the closed circle 13226 is filled with red.

When the glucose concentration falls rapidly, the closed circle 13226 is filled with dark-red.

The above description is only illustrative, and there may be other corresponding relationships between the tendency of glucose and the filling color, so it is not limited here.

In some embodiments of the invention, the filled color of the closed circle 13226 can be used to replace the arrow of the glucose tendency icon 13221, so as to simplify the display content of the second display area 1322. However, the filled color of the closed circle 13226 indicates the tendency of glucose, and the arrow indicates the tendency of glucose intuitively.

In some embodiments of the invention, the color filling to indicate the tendency of glucose can also be used for the glucose concentration unit 13225, the glucose concentration detection time 13223, or the background area of the second display area 1322, and is not limited here.

In some embodiments of the invention, the glucose concentration value 13222 and the glucose concentration unit 13225 are vertically distributed, see FIG. 6a.

In other embodiments of the invention, the glucose concentration value 13222 and the glucose concentration unit 13225 are left and right distributed, see FIG. 6c.

In some embodiments of the invention, since the glucose concentration value 13222 is more important to the user than the glucose concentration unit 13225, the glucose concentration detection time 13223, or the glucose tendency icon 13221, the characters of the glucose concentration value 13222 are larger, which is convenient for the user to read the glucose concentration value 13222.

In some embodiments of the invention, the glucose concentration value 13222 is composed of the integer 13222a and the decile 13222b. In some cases, the user may pay more attention to the integer 13222a, so the character of the integer 13222a is larger than the character of the decile 13222b. See FIG. 6d.

### Indicator Light

FIG. 7 is the schematic diagram of the plurality of quantity indicator lights according to the embodiment of the invention.

Referring back to FIG. 1, in some embodiments of the invention, the indicator light 135 and the screen 132 are located on the same side of the management device 13, so that the user can easily see the light of the indicator light 135.

In some embodiments of the invention, the indicator light 135 is the LED light with changeable color. Under different prompt requirements, the indicator light 135 can flash at different intervals/durations/colors. For example, the corresponding relationship between the indicator light 135 and the prompt may be as follows:

Trigger the high/low glucose alarm prompt, and the indicator light 135 is red, always on and not off.

Prompt for glucose probe calibration: the indicator light 135 turns on yellow, turns on for 1 s, and turns off for 1 s.

The management device 13 prompts that the power is low, and the power of the indicator light 135 is red, which is on for 1 s and off for 2 s.

When the glucose probe signal is lost, the indicator light 135 is yellow, always on and not off. There is no reading prompt for the glucose probe, and the indicator light 135 is yellow, which is on for 1 s and off for 3 s.

The above correspondence is only illustrative and is not limited here.

In other embodiments of the invention, the indicator light 135 may be located on the side or the back of the management device 13.

Referring to FIG. 7, in some embodiments of the invention, the indicator light 135 may be the combination light of two or three or more lights, which may have the same lighting color, and indicate the contents to be prompted by the arrangement of the lighting or deactivating positions of the lights, and/or the interval/duration of the flashing of the lights. In other embodiments of the invention, these lights may have different lighting colors, and the contents to be prompted are indicated by the arrangement of the positions where the lights are lit or deactivated, and/or the interval/duration/color of the flashing of the lights.

### Scroll Bar

FIG. 8 is the schematic diagram of the scroll bar according to the embodiment of the invention.

Due to the size limitation of the screen 132, part of the content to be displayed may not be fully displayed on the screen, and it is necessary to slide the screen up and down or left and right to display the full content.

Referring to FIG. 8, in some embodiments of the invention, the scroll bar 132e1 is provided on the right edge of the screen 132. The user taps the scroll bar 132e1 with his finger and slides up and down the screen 132. The content displayed on the screen 132 can slide up and down with the scroll bar 132e1 to display the complete content.

In some embodiments of the invention, with the sliding of the scroll bar 132e1, the content displayed on the screen 132 slides line by line, that is, one line of characters is slid at the time. In other embodiments of the invention, as the scroll bar 132e1 slides, the content displayed on the screen 132 slides two or more lines, that is, multiple lines of characters are slid each time.

In some embodiments of the invention, the scroll arrow 132e2 is provided on the right edge of the screen 132. The scroll arrow 132e2 comprises upward and downward arrows. Clicking the upward or downward arrows respectively can slide the contents displayed on the screen 132 upward or downward.

In some embodiments of the invention, each time the scroll arrow 132e2 is clicked, the content displayed on the screen 132 is slid line by line, that is, one line of characters is slid at the time. In other embodiments of the invention, each time the scroll arrow 132e2 is clicked, the content displayed on the screen 132 slides two or more lines, that is, multiple lines of characters are slid each time.

### Alarm/Alert Prompt

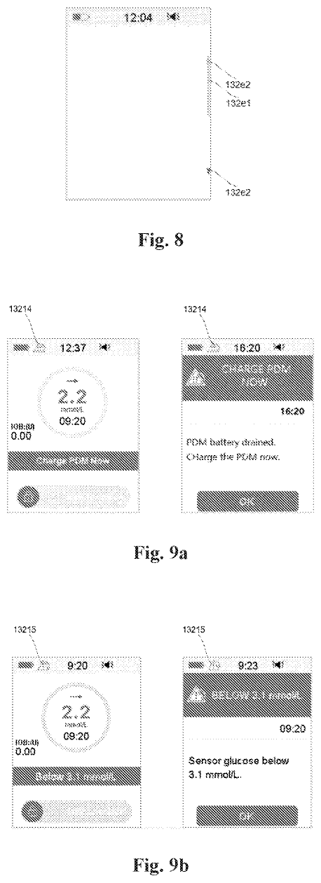

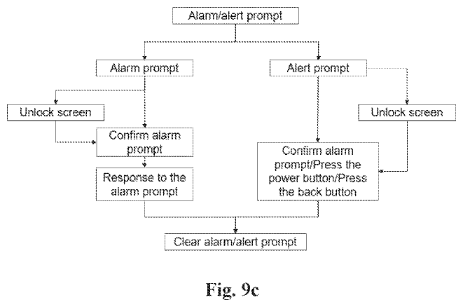

FIG. 9a is the schematic diagram of the alarm prompt according to the embodiment of the invention. FIG. 9b is the schematic diagram of the warning prompt according to the embodiment of the invention. FIG. 9c is the flow diagram of clearing the alarm prompt according to the embodiment of the invention.

In some embodiments of the invention, the alarm prompt may be divided into the alarm prompt with high priority and the alert prompt with low priority.

Referring to FIGS. 9a and 9c, in some embodiments of the invention, the alarm/alert prompt is triggered in the serious or potentially serious situation. When the alarm prompt is triggered, the user must make the corresponding response to clear the alarm. For example, when the management device 13 is about to run out of power, the alarm prompt of “charge the device now” will be triggered, and the text prompt of “charge the device now” will be displayed on the screen 132. After confirming the alarm prompt, the user needs to charge the management device 13 as soon as possible. If the management device 13 is not charged within 10 minutes, the management device 13 will trigger the alarm prompt repeatedly until the user charges the management device 13.

In some embodiments of the invention, when the alarm prompt is triggered, if the screen is in the screen lock state, the user needs to unlock the screen 132 first. Refer to “screen lock and unlock” for specific operations.

Referring to FIGS. 9b and 9c, in some embodiments of the invention, the alert prompt is triggered when attention needs to be paid. When the alert prompt is triggered, the user can press the power button 131 or the home key 133 or make the corresponding response to clear the alert. For example, when the glucose concentration is too low, the hypoglycemia warning prompt will be triggered. The screen 132 displays “below XX mmol/L”. After the user confirms the alert prompt or presses the power button 131 or the home key 133, the hypoglycemia alert prompt will disappear.

In some embodiments of the invention, when the alert prompt is triggered, if the screen is in the screen lock state, the user needs to unlock the screen 132 first. Refer to “screen lock and unlock” for specific operations.

In some embodiments of the invention, when the alarm prompt or the alert prompt appears, the alarm icon ( [symbol] ) 13214 or the alert icon ( [symbol] ) 13215 appears in the first display area 1321, respectively. In the preferred embodiment of the invention, the alarm icon 13214 and the alert icon 13215 may be filled with different colors to facilitate the user to distinguish. For example, the alarm icon 13214 may be filled with red and the alert icon 13215 may be filled with yellow.

In some embodiments of the invention, when the alarm prompt or the alert prompt is triggered, there are different prompt modes, see FIG. 2c. According to the user's selection of the current prompt mode, for example, the user selects the sound prompt. When the alarm/alert prompt is triggered, the management device 13 emits 10 sounds every 20 seconds, and when the alarm/alert prompt is triggered, the management device 13 emits 2 sounds every 3 minutes. For another example, when the user selects the vibration prompt, the management device 13 vibrates once every 20 seconds when the alarm/alert prompt is triggered, and when the alarm/alert prompt is triggered, the management device 13 vibrates once every 3 minutes. For another example, when the user selects sound and vibration prompt, the management device 13 emits sound 10 times every 20 seconds and vibrates once every 20 seconds when the alarm/alert prompt is triggered. When the alarm/alert prompt is triggered, the management device 13 emits sound twice every 3 minutes and vibrates once every 3 minutes. For another example, when the user selects to turn off the sound and vibration prompt and triggers the alarm prompt or the alert prompt, the management device 13 will neither emit sound nor vibrate. The above description is only exemplary, and the frequency of sound or vibration is not limited here.

In summary, the embodiments of the invention disclose a management device for displaying glucose information. The screen of the management device is divided into the plurality of display areas, comprising at least a first display area displaying the status of the management device, a second display area displaying real-time glucose information, and a third display area displaying historical glucose concentration information, the user can intuitively and conveniently get the glucose information in the body through the home screen of the management device.

Although some specific embodiments of the invention have been detailed through examples, technicians in the field should understand that the above examples are for illustrative purposes only and are not intended to limit the scope of the invention. Persons skilled in the field should understand that the above embodiments may be modified without departing from the scope and spirit of the invention. The scope of the invention is limited by the attached claims.

1. A management device for displaying glucose information, comprising:
a screen capable of user interaction, wherein the screen is divided into a plurality of display areas, wherein the plurality of display areas at least comprises a first display area for displaying a status of the management device, a second display area for displaying real-time glucose information, and a third display area for displaying historical glucose concentration information, wherein at least one display area further comprises calibration information;
a power button, wherein the power button is at least used to activate or deactivate the screen;
and a home key, wherein the home key is at least used to return an interface displayed on the screen to a home screen.

2. The management device of claim 1, wherein the first display area comprises at least a power icon, a time icon and a prompt mode icon.

3. The management device of claim 2, wherein the power icon, the time icon and the prompt mode icon have different display forms under different operation or setting states.

4. The management device of claim 1, wherein the second display area at least comprises a glucose tendency icon, a glucose concentration value, a glucose concentration detection time and a closed circle, and the glucose tendency icon, the glucose concentration value and the glucose concentration detection time are located in the closed circle.

5. The management device of claim 4, wherein a tendency of glucose is represented by a trend icon.

6. The management device of claim 5, wherein the trend icon is filled with a suggestive color.

7. The management device of claim 4, wherein a tendency of glucoses is represented by coded color.

8. The management device of claim 7, wherein the coded color is filled in a background area of the closed circle or the second display area.

9. The management device of claim 4, wherein when an event is triggered, at least one of the glucose tendency icon, the glucose concentration value, and the glucose concentration detection time is replaced by an event icon.

10. The management device of claim 9, wherein the event at least comprises a warm-up of glucose probe, a glucose probe calibration, a loss of signal, an abnormal glucose probe detection or an abnormal glucose concentration value.

11. The management device of claim 1, wherein the third display area comprises a dynamic graph with time as a horizontal axis and glucose concentration value as a vertical axis, wherein the dynamic graph is for displaying historical glucose information.

12. The management device of claim 11, wherein a real-time glucose concentration value is located at a right most side of the dynamic graph.

13. The management device of claim 11, wherein when the user taps the dynamic graph, an information display associated with the historical glucose information is expanded in the dynamic graph.

14. The management device of claim 1, wherein the second display area and the third display area update display at fixed time intervals.

15. The management device of claim 14, wherein the fixed time interval is 2 min.

16. The management device of claim 1, wherein when an alarm event is triggered, an alarm interface replaces at least one of the plurality of display areas of the home screen.

17.-47. (canceled)
